(19) United States
(12) Patent Application Publication
(10) Pub. No.: US 2023/0193197 A1
(43) Pub. Date: Jun. 22, 2023

(54) TUNABLE PLANT-BASED MATERIALS VIA IN VITRO CELL CULTURE USING A ZINNIA ELEGANS MODEL

(71) Applicant: FORAY Bioscience, Inc., Cambridge, MA (US)

(72) Inventor: Ashley Lynne BECKWITH, Cambridge, MA (US)

(73) Assignee: FORAY Bioscience, Inc., Cambridge, MA (US)

(21) Appl. No.: 18/061,095

(22) Filed: Dec. 2, 2022

Related U.S. Application Data
(60) Provisional application No. 63/292,067, filed on Dec. 21, 2021.

(51) Int. Cl. C12N 5/00; C12N 5/04

(52) U.S. Cl. CPC C12N 5/0025; C12N 5/04

(57) ABSTRACT

The process described herein may provide the benefit of selectively generating plant-based materials with tunable cellular compositions and material properties in controlled forms without necessarily requiring whole-plant cultivation or harvest. An example process may include extracting and maintaining live plant cells via leaf maceration and liquid culturing, transferring cells from the liquid culture to a gel medium, integrating the cells into a hydrogel scaffold, and shaping the scaffold. This process, using the disclosed tissue engineering-style approach, may further allow for localized and high-density biomass production, eliminate energy intensive harvest and hauling, reduce processing, and inherently foster climate resilience.

### CROSS-REFERENCE TO RELATED APPLICATIONS

This application claims priority to U.S. Provisional Application No. 63/292,067, filed Dec. 21, 2021, the entirety of which is incorporated herein by reference.

### BACKGROUND

Each year, global forests lose billions of trees as a result of human activities and natural disasters. This sustained deforestation impacts both environment and economy. Forests are ecologically essential—supporting biodiversity, stabilizing ecosystems, and sequestering carbon. Meanwhile, trees also supply feedstock for building infrastructure, energy generation, production of consumer goods, textile manufacturing, and an increasing range of other economic activities.

Plant-based feedstock production has changed little in centuries: whole plants are cultivated, useful portions are harvested, and the remaining portions are discarded or burned for energy. Useful fractions of the original biomass are then mechanically or chemically restructured into functional forms or isolated chemical compounds (e.g., cellulose). With surging demands for plant-based feedstocks, efforts have been made, on a process-specific level, to reduce inefficiencies of biomass manipulation within the industrial setting. Nevertheless, the greatest costs and resource consumption in the supply chain often precede these steps, e.g., significant amount of time, land, water, fertilizers, and pesticides are dedicated to cultivation of whole plants. In addition, harvest and transportation of biomass to processing locations may involve significant investment of financial capital and energy (e.g., in harvesting woody biomass, logging and transportation expenses make up a sizeable fraction of gate costs. Despite considerable and early resource investment, only a small fraction of the cultivated crop may be economically valuable at harvest. For the production of some natural fibers, as little as 2%-4% of the harvested plant matter comprises useful material; for other crops, just one third of the stem dry weight may be characterized as such. Therefore, conventional plant-based feedstock production remains wasteful and expensive.

### SUMMARY

Embodiments disclosed herein attempt to solve the aforementioned technical problems and may provide other solutions as well. In an example, a process of generating tunable plant-based materials is provided. The process may include extracting and culturing live plant cells via liquid medium, transferring the cultured cells to a nutrient rich gel medium, integrating the cells into a hydrogel scaffold, and shaping the scaffold via casting, bioprinting, or molding. Therefore, the process may provide the benefit of selectively generating plant-based materials with tunable cellular compositions and material properties in controlled forms without necessarily requiring whole-plant cultivation or harvest. This process, using the disclosed tissue engineering-style approach, may further allow for localized and high-density biomass production, eliminate energy intensive harvest and hauling, reduce processing, and inherently foster climate resilience.

In an embodiment, a method of generating plant-based biomass is provided. The method may include selectively extracting and maintaining live plant cells via leaf maceration and liquid culturing; transferring cells from a liquid culture to a gel medium and integrating the cells into a hydrogel scaffold; and shaping the scaffold.

### BRIEF DESCRIPTION OF THE DRAWINGS

FIG. 1 shows an example process flow of selective tissue-like growth from a non-destructive plant sample, according to some embodiments of this disclosure.

FIG. 2A shows an example chart illustrating a characterization of cell response to varied hormone levels using live fraction, cumulative lignification metric, enlargement metric, and elongation metric, according to some embodiments of this disclosure.

FIG. 2B shows an example chart illustrating a characterization of cell response to varied hormone levels using cumulative lignification metric, according to some embodiments of this disclosure.

FIG. 2C shows an example chart illustrating a characterization of cell response to varied hormone levels using enlargement metric, according to some embodiments of this disclosure.

FIG. 2D shows an example chart illustrating a characterization of cell response to varied hormone levels using elongation metric, according to some embodiments of this disclosure.

FIG. 3 shows an example graph depicting live fraction versus time in pH-adjusted culture, according to some embodiments of this disclosure.

FIG. 4 shows an example graph depicting lignification versus time in pH-adjusted culture, according to some embodiments of this disclosure.

FIG. 5A shows an example graph depicting cell enlargement metric versus time in pH-adjusted culture, according to some embodiments of this disclosure.

FIG. 5B shows an example graph depicting elongation metrics versus time in pH-adjusted culture, according to some embodiments of this disclosure.

FIG. 6A shows an example graph depicting enlargement metrics versus initial cell concentration, according to some embodiments of this disclosure.

FIG. 6B shows an example graph depicting elongation metrics versus initial cell concentration, according to some embodiments of this disclosure.

FIG. 7 shows an example border of a confluent, casted gel-based culture with elongated cells, according to some embodiments of this disclosure.

FIG. 8 shows an example of confluent, bioprinted culture with lignified cells, according to some embodiments of this disclosure.

### DESCRIPTION

Conventional plant-based biomass production may involve cultivation of whole plants, which is time consuming and wasteful, often involving rampant deforestation across the globe. To solve this dilemma, the present disclosure provides processes, systems, and methods for reducing waste, saving time, and protecting forests across the globe by selectively generating plant-based biomass from isolated plant cells. An example process may include extracting and isolating live plant cells to establish a liquid suspension culture, transferring cells from a liquid medium used for the low hormone liquid culturing to a nutrient rich gel medium and thus integrating the cells into a hydrogel scaffold, and shaping the scaffold via casting, bioprinting, or molding. Therefore, the process provides the benefit of selectively generating plant-based materials without necessarily requiring whole-plant cultivation or harvest, allowing for high-density production, eliminating energy intensive harvest and hauling, reducing processing, and inherently fostering climate resilience.

Described herein are examples of parameters including hormone concentrations, medium pH, and initial cell density which may quantifiably influence cell development and morphology. Differences in cellular-level culture characteristics may then be related to changes in final material properties demonstrating the tunability of grown materials at cellular and macroscopic scales. Control over cellular-level shape is enabled by imparting developmental cues (e.g., hormones) which can influence the shape, size, identity of the cells.

Control over material form may be made possible by the casting, bioprinting, or molding of cell laden scaffolds, illustrating the potential of near-net-shape plant material production. Control over material form may be made possible by the casting, bioprinting, or molding of cell laden scaffolds, illustrating the potential of near-net-shape plant material production. Scaffolds are engineered materials which may mimic the native environment to the cell or tissue cultured on them. In an embodiment, cell doped gel media solution may be printed into a cell-doped scaffold using a bioprinter such as, e.g., a Tissue Scribe 3D bioprinter. In another embodiment, cultivated materials may be cast or molded into a cell-doped scaffold.

It should be understood that the specific numerical values recited in the embodiments below are merely provided as examples and should not be considered limiting. Embodiments with different numerical values should also be considered within the scope of this disclosure.

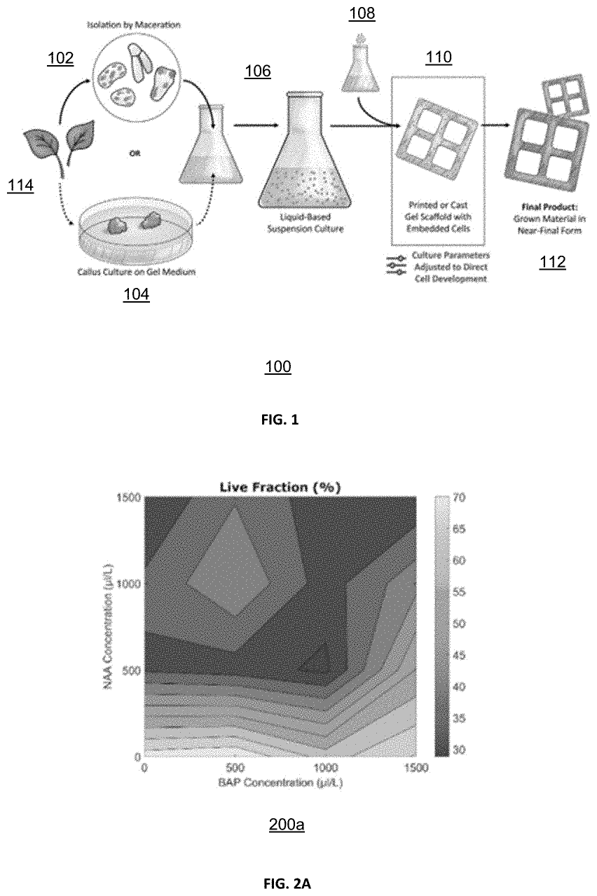

FIG. 1 shows an example process flow 100 of selective tissue-like growth from a non-destructive plant sample 114, according to some embodiments of this disclosure. In some aspects, Zinnia elegans (“Z. elegans”) cells may be isolated through maceration of young leaves 102. In other aspects, other species may use an intermediate callus culture step, whereby a cell mass may be initiated through prolonged culture atop a gelled, nutrient-rich medium 104. In both cases, collected cells may be transferred to a liquid culture 106 where they may be cultivated, sub-cultured, and utilized as a long-term feedstock for the subsequent culture steps.

In an embodiment, Z. elegans cells may be isolated by the maceration of young Zinnia leaves (e.g., leaves 114). Leaves collected from approximately 14 day-old Zinnia plants may be rinsed under running tap water for 5 min. Leaves may be sterilized in a solution of 5 ml commercial bleach (Clorox), 95 ml of deionized water, and 200 ml of Tween 20 (Sigma Aldrich) for 5 min. Subsequently, leaves may be rinsed thoroughly with sterile distilled water, sliced into strips, and ground between the surfaces of a small sieve and a spoon (e.g., a stainless steel spoon). The leaf matter may be intermittently rinsed with ZE-M media and the rinsate may be collected in a bowl positioned beneath the supporting sieve. After completion of grinding, the rinsate may be collected and filtered through a 70 mm cell strainer (e.g., manufactured by Fisher Scientific) to remove large debris. This strainer is just but an example and should not be considered limiting. The filtered solution may be centrifuged at 100 g for 8 min and resulting supernatant removed if more concentrated cell solutions are desired. Liquid cultures may be maintained at concentrations between 250,000 and 500,000 cells ml⁻¹ at 3 ml per well in a 6-well plate wrapped in parafilm. Cultures may be maintained at 22° C. in the dark on an orbital shaker operating at 80 rpm.

Cells may be cultured in this manner for 48 hours in the low-hormone media before transference to specialized experimental media. An incubation period may improve differentiation rates when cells may be later exposed to elevated levels of auxin and cytokinin. Auxins may be plant hormones produced in the stem tip that promote cell elongation. Cytokinins may be a class of plant hormones that promote cell division, or cytokinesis. For dispersed gel cultures, cells may be extracted and maintained in low-hormone, liquid medium for a 48 hour acclimation period, as previously described, prior to transference to a gel medium 108 solidified by the addition of Gelzan C M (e.g., manufactured by Sigma Aldrich) at a final concentration of 3 g L⁻¹. Cultures may be sealed off with parafilm and maintained at 22° C. in the dark.

In another embodiment, additional tree species including but not limited to Pinus radiata or Populus Trichocarpa, or non-tree species including but not limited to Nicotiana tabacum, or Arabidopsis thaliana may be isolated either via leaf maceration as described above or via an intermediate callus culture step, whereby a cell mass may be initiated through prolonged culture atop a gelled, nutrient-rich medium.

To initiate construct growth, cell suspension stock may be mixed with a thermosetting gel medium at a 1:3 ratio (v/v). The resulting mixture may solidify when cooled to room temperature to yield a culture of single cells dispersed with a gelled, nutrient-rich scaffold 110. With time, the dispersed gel cultures may grow to generate confluent cellular material 112. In gel mediated culture, cells may survive for several weeks and, by tuning local biochemical and mechanical properties, cells may be directed to develop into desirable cell types or morphologies. The shape of the cultivated materials may be controlled via casting, bioprinting (e.g., by Tissue Scribe Gen.3, 3D Cultures), or molding of cell-doped scaffolds.

For liquid cultures, well-mixed 250 ml aliquots of cell suspension may be transferred to 48 well plates for imaging. A 5 ml volume of a fluorescein diacetate (FDA) stock solution (prepared at 2 mg ml⁻¹ acetone) may be added to the cell suspension and incubated in the dark for 20 min. After the incubation period, 63 ml of calcofluor white (CW) may be added and the solution rested for an additional 5 min prior to imaging. A Zeiss LSM780 confocal microscope, for example, may be set to excitation/emission wavelengths of 265 nm/440 nm (DAPI filter) for CW and 490 nm/526 nm for FDA for imaging. Gel cultures may be stained through a similar dual-step process after wetting the gel surface with a small amount of liquid medium. Relative staining volumes may be the same, but increased incubation times may be involved (e.g., incubation times were approximately 45 min for FDA incubation, 45 min for CW incubation). Image analysis may be performed using Image-i. The thresholding tool may be used to select the relevant cell areas. To ensure robustness of the analyzed data ranges, the images corresponding to the highest and lowest values for a single sample may be analyzed a total of 3 times, with the repeated results averaged to yield a final value for the given image.

In plants, cellular make-up of a plant tissue may affect the corresponding macroscopic mechanical properties. For example, elevated proportions of highly aligned, stiffened (i.e., with a lignified secondary cell wall) vascular cells in plant stems may contribute to increased rigidity of the tissue. Therefore, understanding and controlling cellular composition of cultured biomaterials may be useful to producing useful substances for a wide range of applications. The study of cellular development in response to culture parameters may allow for growth optimization yielding substrates with desirable compositions of cellular constituents and associated macro-scale material properties. Numerous environmental factors may impact plant cell development in vitro. The disclosed embodiments may particularly control the parameters such as the interactive effects of two hormone concentrations, medium pH, and initial cell density. These parameters may allow for measurable and largely independent manipulation, and the cells may be responsiveness to their adjustments.

Although hormone concentration, pH, cell density and other factors may be independently investigated to various extents, thorough re-evaluation of these variables may be desired in order to, for example: (a) verify relevant cell behavior in spite of new, simplified media recipes (Tables 1 and 2), (b) characterize previously unreported developmental traits such as enlargement and elongation, (c) demonstrate the use of new metrics to quantify cell development, and (d) contribute to the limited information available on cell growth and development in dispersed gel culture. Four example measurement metrics, e.g., live fraction, lignification metric, enlargement metric, and elongation metric, may quantify collective culture development in a measurable way and allow for a selection of culture parameters to suit output requirements. The metrics may be tabulated from micrographs and reflect live fraction, tracheary element differentiation (e.g., lignification), cell enlargement, and/or cell elongation in response to applied culture conditions.

TABLE 1
Recipe for Z. elegans maintenance medium (ZE-M).

| | | Quantity |
|---|---|---|
| | | [per liter of |
| Product name | Supplier | medium] |
| N616 Nitsch medium | Phytotech Laboratories | 2.21 g |
| Sucrose | Sigma Aldrich | 10 g |
| Mannitol | Sigma Aldrich | 36.4 g |
| [symbol] -Naphth [symbol] acetic acid (NAA) | Sigma Aldrich | 0.001 mg [symbol] |
| [symbol] -Benzylaminopurine | Sigma Aldrich | 1 μl |
| solution (BAF) | | |

[symbol]

indicates data missing or illegible when filed

TABLE 2
Recipe for Z. elegans induction medium (ZE-1) for differentiation.

| | | Quantity |
|---|---|---|
| | | [per liter of |
| Product Name | Supplier | medium] |
| N616 Nitsch medium | Phytotech Laboratories | 2.21 g |
| Sucrose | Sigma Aldrich | 10 g |
| Mannitol | Sigma Aldrich | 36.4 g |
| [symbol] -Naphth [symbol] acetic acid (NAA) | Sigma Aldrich | 1 mg [symbol] |
| [symbol] -Benzylaminopurine | Sigma Aldrich | 1 ml |
| solution (BAF) | | |

[symbol]

indicates data missing or illegible when filed

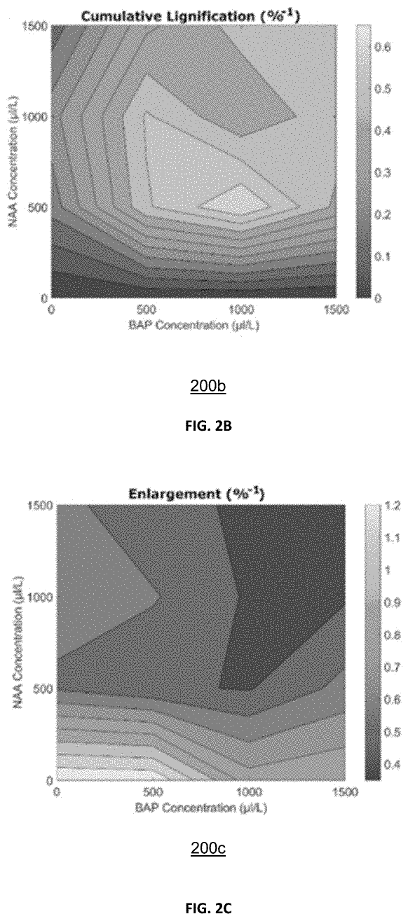

FIG. 2A shows an example chart 200 illustrating a characterization of cell response to varied hormone levels using a live fraction metric, according to some embodiments of this disclosure. The live fraction metric may be the ratio between the percentage of the micrograph occupied by cells marked with a viability probe (fluorescein diacetate), A_{L}, and the percentage of the micrograph occupied by all cells marked with a cell wall stain (calcofluor white), A_{T}, i.e., Live Fraction [%]=100%*A_{L}/A_{T}; (1). Live fraction may provide insights into cell health and may act as a secondary indicator of tracheary element differentiation as cells undergo programmed cell death at late stages of development.

FIG. 2B shows an example chart 200b illustrating a characterization of cell response to varied hormone levels using cumulative lignification metric, according to some embodiments of this disclosure. The lignification metric may be the ratio between C_{L}—the number of lignified cells in the micrograph, and the corresponding A_{T}, i.e., Lignification Metric [%⁻¹]=C_{L}/A_{T}. The lignification metric may further quantify the extent of culture differentiation into tracheary elements possessing a rigid, lignified cell wall, the presence of which may increase stiffness of the overall grown material.

FIG. 2C shows an example chart 200c illustrating a characterization of cell response to varied hormone levels using enlargement metric, according to some embodiments of this disclosure. The cell enlargement metric may be the ratio between C_{En}—the number of cells in the micrograph with a maximum dimension greater than a certain threshold, and the corresponding A_{T}, i.e., Cell Enlargement Metric [%⁻¹]=C_{En}/A_{T}; The threshold value for cell enlargement, I_{α}, may represent the maximum dimension of cultured cells at 48 h after cell isolation (la is approximately equal to 84 mm in this case). The cell enlargement metric may provide an indicator of average cell-level growth or swelling.

FIG. 2D shows an example chart 200d illustrating a characterization of cell response to varied hormone levels using elongation metric, according to some embodiments of this disclosure. The cell elongation metric may be the ratio between C_{El}—the number of cells in the micrograph with a maximum dimension greater than a certain threshold, and the corresponding A_{T}, i.e., Cell Elongation Metric [%⁻¹]=C_{El}/A_{T} I threshold value for cell elongation, I_{β}, may represent the maximum dimension of cells grown in low-hormone media for 12 days that exhibit multi-directional enlargement without pronounced uniaxial elongation (I_{β} may be approximately equal to 119 mm). Greater proportions of elongated cells may increase prevalence of cell-to-cell entanglement in confluent cultures, potentially influencing grown material properties. For viability metrics and the calculation of percent cell area (A_{T}) in all cases, two-channel images visualizing FDA and CW may be taken with focal plane adjusted to resolve FDA features. Lignified cell counts, enlargement, and elongation measurements may be made on a corresponding single-channel CW image, with identical field of view but focus adjusted slightly to resolve CW features.

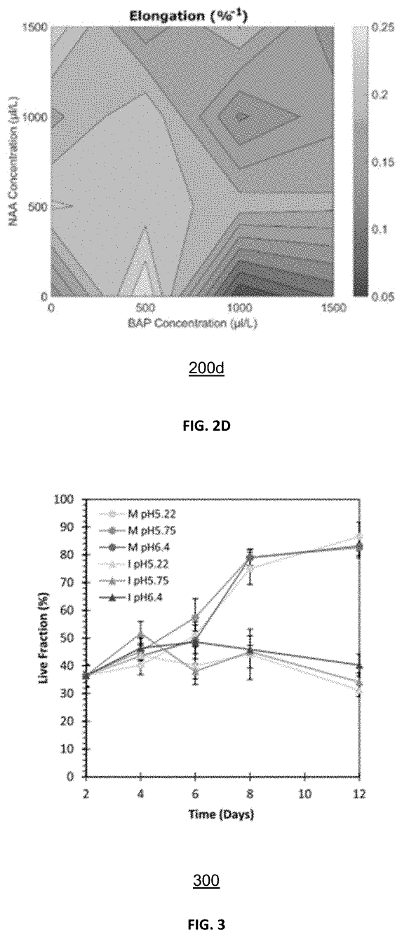

Generally, data reported for a specific timepoint and treatment may be averaged across all images evaluated for that treatment on that day. Error bars on provided data plots (e.g., as shown in FIGS. 3, 4, 5A, 5B, 6A, and 6B) may represent one sample standard deviation above and below the mean. Two sample t-tests may be performed at a confidence level of 95% to establish P-values between pairs of datasets (Matlab). In the case of hormone response experiments, in which cell response to two factors may be investigated simultaneously, a full factorial design may be performed at 4, near-equally-incremented levels of each hormone concentration (i.e., amounting to 16 individual hormone combinations). Factorial approaches may generally be preferred to one-factor-at-a-time experimentation because they may enable the detection of interaction effects between variables. The resulting response metrics may be mapped using the Matlab contour function.

An example of the effects of two hormone classes, i.e., auxin and cytokinin, on plant cell development may be seen in FIG. 2A-D. Both auxin and cytokinin may be critical to vascular tissue development in particular and independently control a wide range of cell behaviors. Considered together, the hormones may elicit complex, interactive effects.

For example, elevated levels of both auxin and cytokinin may induce the differentiation of Z. elegans cells into lignified tracheary elements, while collectively low hormone concentrations may be provided to Z. elegans cultures to encourage maintenance and proliferation without further differentiation. Cell morphology and development at unbalanced ratios of auxin and cytokinin concentrations may not be as well-characterized, particularly in relation to cell enlargement and elongation. According to one or more embodiments of this disclosure, a full factorial experiment may be performed to evaluate cellular development at a range of hormone concentrations over a total 12-day culture period, using the previously described metrics. After a 48 hour acclimation phase in which isolated cells may be cultured in low hormone media, samples may be transferred to treatment media and imaged periodically over the course of the subsequent 10 days. Hormones selected for evaluation may include two commonly employed in plant cell culture: a synthetic auxinda-naphthaleneacetic acid (NAA), and a synthetic cytokinind6-benzylaminopurine solution (BAP). Hormone concentrations may range from 0.001 mg ml⁻¹, as recommended for culture maintenance, to 1.5 mg ml⁻¹, one and a half times the concentration regularly cited for tracheary element induction, evaluated at approximately 0.5 mg ml⁻¹ intervals. Other hormones may also be selected including, but not limited to, kinetin, 2,4-Dichlorophenoxyacetic acid, Zeatin, or indoleacetic acid.

The experimental results may demonstrate that tuning hormone concentrations may allow for control over final cellular composition of the treated culture. In low hormone media, cells may exhibit high levels of viability (>70% live fraction) after ten days in treatment media and the corresponding levels of lignification may remain at or near zero. Cells in low-hormone treatment media may enlarge over time, but experience may limit uniaxial elongation. The highest cumulative levels of lignification may occur with NAA at 0.5 mg ml⁻¹ (0.5 ml L⁻¹ of stock solution) and BAP at 1 mg ml⁻¹ (1 ml L⁻¹ of stock solution); correspondingly, these hormone levels may exhibit the lowest live fraction at day 12, as lignifying tracheary elements undergo programmed cell death in the final stages of differentiation. For this reason, the lignification metric may trend inversely with live fraction.

Both the day-to-day and cumulative lignification values (as shown in FIG. 2B) may align with this projected behavior. Results may also indicate that with the selected base media formulation, BAP plays an important role in determining the elongation fate of cells, although this control may be commonly attributed to auxin specifically. Elongation metric plotted across hormone levels may show that when BAP is elevated, elongation tends to be reduced across most NAA concentrations (as shown in FIG. 2D).

Auxin-mediated cell elongation may be believed to act, at least in part, by encouraging the release of cell wall-loosening factors, which may include hydrogen ions. Wall-loosening factors may be believed to promote wall compliance, enabling cell expansion and restructuring. Therefore, hydrogen ion concentration in the growth medium, as reflected by pH, may similarly be suspected to influence cellular development.

FIG. 3 shows an example graph 300 depicting live fraction versus time in pH-adjusted culture, according to some embodiments of this disclosure. To quantify the effects of pH on cell development, Z. elegans cells may be cultured in either maintenance media (ZE-M) or induction media (ZE-I) at one of 3 pH values (i.e., prepared at pH 5.22, 5.75, or 6.4 prior to a final autoclave sterilization). After a 48 h acclimation period in which all isolated cells may be cultured in low-hormone medium at pH 5.75, samples may be transferred to treatment media and imaged periodically over the course of the subsequent 10 days. From the analysis of fluorescence micrographs, live fraction, lignification, enlargement, and elongation metrics may be evaluated for each of the culture treatments. In maintenance media cultures, pH may have negligible effect on live fraction or lignification metrics. For all pH levels, live fraction for ZE-M cultures may increase from a starting point of 36.4% to a final value close to 80%. These results may trend similarly with those seen in the factorial hormone experiment where low-hormone cultures may experience a shift in live fraction from 36.8% to an excess of 70% at the final time-point.

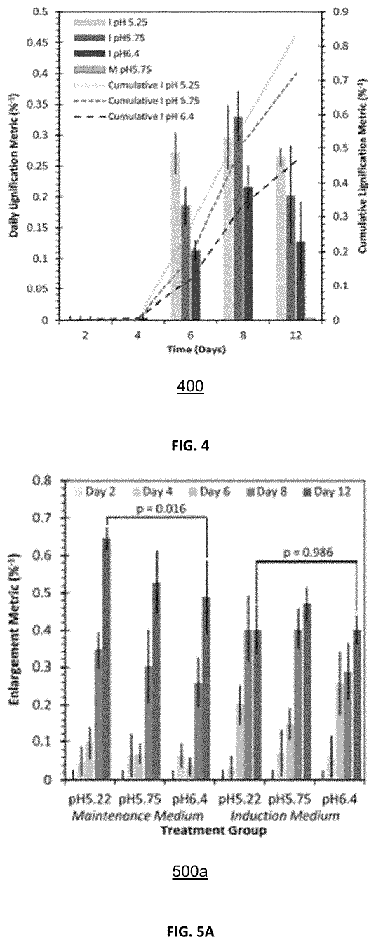

FIG. 4 shows an example graph 400 depicting lignification versus time in pH-adjusted culture, according to some embodiments of this disclosure. For the pH evaluation, the lack of significant differences between live fraction of maintenance media (ZE-M) groups may suggest that the selected pH values may not be independently detrimental to cell viability. At low hormone levels and low pH levels, measured cells may tend to be larger in size as quantified by enlargement and elongation metrics. Low pH ZE-M samples may be significantly larger than medium or high pH samples (P=0.03 and 0.016, respectively). Low pH ZE-M samples may exhibit greater elongation than moderate pH samples (P=0.018) which in turn, may experience significantly greater elongation than high pH samples (P=0.0095) by day 12. For cell cultures grown in high-hormone media (ZE-I), pH may prove to be influential in lignification and live fraction metrics. Low pH, ZE-I cultures may present significantly elevated lignification metrics for days 6, 8, and 12 when compared to high pH samples (P=0.00072, 0.045 and 0.0013, respectively). Inversely, live fraction for low pH groups may be significantly lower than those in high pH samples on the final culture day (P=0.00062). These results may align with the hypothesized inverse relationship between differentiation and live fraction.

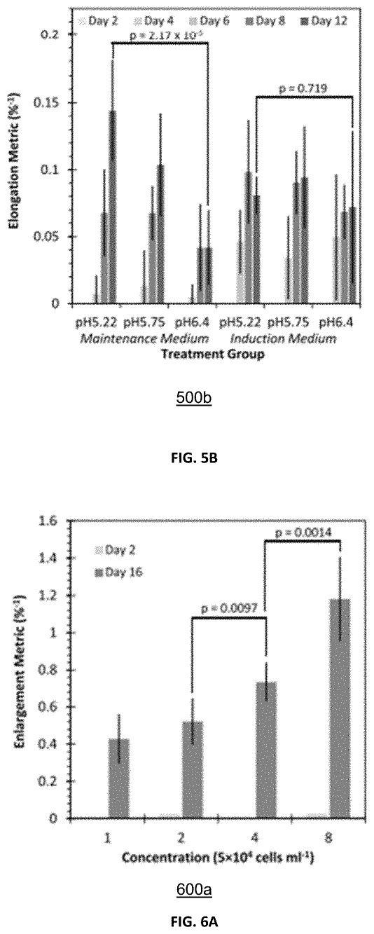

FIG. 5A shows an example graph 500a depicting cell enlargement metric versus time in pH-adjusted culture, according to some embodiments of this disclosure. FIG. 5B shows an example graph 500b depicting elongation metrics versus time in pH-adjusted culture, according to some embodiments of this disclosure. While pH may strongly influence enlargement and elongation in the low-hormone maintenance media, pH adjustments may not generate significant trends in morphological development for high-hormone induction cultures.

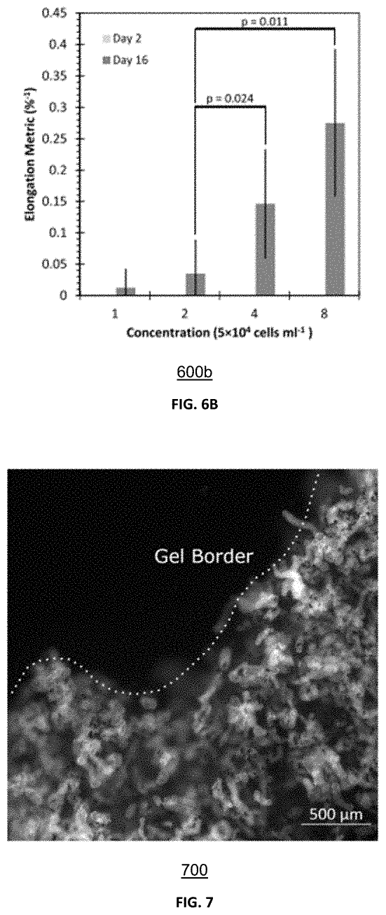

FIG. 6A shows an example graph 600a depicting enlargement metrics versus initial cell concentration, according to some embodiments of this disclosure. FIG. 6B shows an example graph 600b depicting elongation metrics versus initial cell concentration, according to some embodiments of this disclosure. Cell concentration may be reported to influence cellular development in liquid cultures. Thus, the examination of cell density effects on cell morphologies in gel-media may also be necessary to achieve effective control over gel-based culture development. Effects of cell density on development may be quantified through the image analysis of gel cultures established at four starting cell densities and then monitored for a 14-day incubation period. Before the 14-day incubation period, the cells may undergo a 48-hour incubation in low hormone media.

Gel cultures may be prepared at initial cell densities of 5×10⁴, 1×10⁵, 2×10⁵, and 4×10⁵ cells ml⁻¹ (i.e., 1×, 2×, 4×, and 8× multiples of 5×10⁴ cells ml⁻¹). For every time-point, two replicate gel cultures may be prepared at each concentration and at least three independent images may be captured and evaluated per replicate. After 14 days in culture, cells seeded at higher initial cell densities may exhibit increased cell size as quantified by both enlargement and elongation metrics.

While differences between enlargement metrics at low cell densities (i.e., 5×10⁴ cells ml⁻¹(1×) and 1×10⁵ cells ml⁻¹(2×) cultures) may not be significant, metrics for 2×10⁵ cells ml⁻¹(4×) cultures may not be higher than 1×10⁵ cells ml⁻¹(2×) cultures (P=0.0097), and cells of 4⁵ 10⁵ cells ml⁻¹(8×) cultures may be larger than 2×10⁵ cells ml⁻¹ (4×) cultures (P=0.0014). Similarly, with respect to elongation, 5×10⁴ cells ml⁻¹(1×) and 1×10⁵ cells ml⁻¹(2×) values may not be significantly different, but 2×10⁵ cells ml⁻¹ (4×) and 4×10⁵ cells ml⁻¹(8×) cultures may contain longer cells than the 1×10⁵ cells ml⁻¹(2×) cultures as measured by elongation metrics (with P=0.0241 and 0.011, respectively). To confirm that the imaging methods may provide a representative snapshot of the gel cultures in spite of their three-dimensional nature, total evaluated percent area (A_{T}) may be plotted against concentration factor to check for linearity. The relationship between cell concentration and evaluated area may be confirmed to be linear with R-squared values for both time-points exceeding 0.98 when linear trendline intercepts were set to zero; setting the intercept as such may reflect the state at which no cells are present and, therefore, the total percent cell area, A_{T}, may be zero.

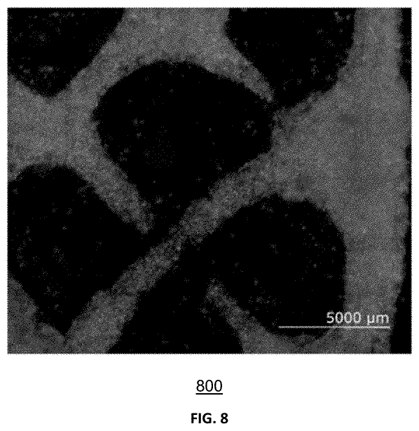

FIG. 7 shows an example border of a confluent, casted gel-based culture 700 with elongated cells, according to some embodiments of this disclosure. FIG. 8 shows an example of confluent, bioprinted culture 800 with lignified cells, according to some embodiments of this disclosure. Insights from the experiments on hormone concentration, medium pH, and cell density may guide the development of lab-grown, plant-based materials. Gel media parameters may be selected based upon the ultimate desired cellular constituents. Macroscopic culture architectures may be controlled either through casting or through 3D bioprinting of a cell-doped gel media solution. Because nutrients and hormones may be incorporated within the scaffold itself, this fully contained setup may involve little intervention after deposition. The scaffold may sustain growth through differentiation and to confluency without requiring supporting perfusion systems. Because viability may not be involved beyond the point of confluency, this approach may provide a simple, low energy means of cultured plant material production.

It should however be understood that the specific numerical parameters above are mere examples, and other numerical parameters should also be considered within the scope of this disclosure.

It will be appreciated by those skilled in the art that the present disclosure can be embodied in other specific forms without departing from the spirit or essential characteristics thereof. The presently disclosed embodiments are therefore considered in all respects to be illustrative and not restricted. The scope of the disclosure is indicated by the appended claims rather than the foregoing description and all changes that come within the meaning and range and equivalence thereof are intended to be embraced therein.

It should be noted that the terms “including” and “comprising” should be interpreted as meaning “including, but not limited to”. If not already set forth explicitly in the claims, the term “a” should be interpreted as “at least one” and “the”, “said”, etc. should be interpreted as “the at least one”, “said at least one”, etc. Furthermore, it is the Applicant's intent that only claims that include the express language “means for” or “step for” be interpreted under 35 U.S.C. 112(f). Claims that do not expressly include the phrase “means for” or “step for” are not to be interpreted under 35 U.S.C. 112(f).

What is claimed is:

1. A method of generating plant-based biomass, comprising:
selectively extracting and maintaining live plant cells via leaf maceration and liquid culturing;
transferring cells from a liquid culture to a gel medium and integrating the cells into a hydrogel scaffold; and
shaping the scaffold.

2. The method of claim 1, wherein extracting the plant cells comprises:
extracting the plant cells from tree species comprising Pinus radiata or Populus trichocarpa, or non-tree species comprising Zinnia elegans, Nicotiana tabacum or Arabidopsis thaliana.

3. The method of claim 1, wherein the cell density of the liquid culture comprises a range of 2×10⁵ ml⁻¹ and 4×10⁵ ml⁻¹.

4. The method of claim 1, wherein the liquid culture comprises a pH range of 5.25-6.5.

5. The method of claim 1, wherein the liquid culture is low hormone liquid culture.

6. The method of claim 5, wherein the low hormone liquid culture comprises synthetic auxin alpha-naphthaleneacetic acid, synthetic cytokinin 6-benzylaminopurine solution, kinetin, 2,4-Dichlorophenoxyacetic acid, Zeatin, or indoleacetic acid.

7. The method of claim 5, wherein the hormones of the low hormone liquid culture comprise a range of 0.001 mg ml⁻¹ and 1.5 mg ml^{-1m}.

8. The method of claim 1, further comprising:
maintaining the cells in the liquid culture for up to 48 hours.

9. The method of claim 1, wherein the hydrogel scaffold is nutrient rich.

10. The method of claim 1, wherein the scaffold is shaped via casting, bioprinting, or molding.

11. A method of generating plant-based biomass, comprising:
selectively extracting and maintaining live plant cells via callus culture and liquid culturing;
transferring cells from a liquid culture to a gel medium and integrating the cells into a hydrogel scaffold; and
shaping the scaffold.

12. The method of claim 11, wherein extracting the plant cells comprises:
extracting the plant cells from tree species comprising Pinus radiata or Populus trichocarpa, or non-tree species comprising Zinnia elegans, Nicotiana tabacum or Arabidopsis thaliana.

13. The method of claim 11, wherein the cell density of the liquid culture comprises a range of 2×10⁵ ml⁻¹ and 4×10⁵ ml⁻¹.

14. The method of claim 11, wherein the liquid culture comprises a pH range of 5.25-6.5.

15. The method of claim 11, wherein the liquid culture is low hormone liquid culture.

16. The method of claim 15, wherein the low hormone liquid culture comprises synthetic auxin alpha-naphthaleneacetic acid, synthetic cytokinin 6-benzylaminopurine solution, kinetin, 2,4-Dichlorophenoxyacetic acid, Zeatin, or indoleacetic acid.

17. The method of claim 15, wherein the hormones of the low hormone liquid culture comprise a range of 0.001 mg ml⁻¹ and 1.5 mg ml^{-1m}.

18. The method of claim 11, further comprising:
maintaining the cells in the liquid culture for up to 48 hours.

19. The method of claim 11, wherein the hydrogel scaffold is nutrient rich.

20. The method of claim 11, wherein the scaffold is shaped via casting, bioprinting, or molding.
